(19) United States
(12) Patent Application Publication
(10) Pub. No.: US 2009/0092547 A1
(43) Pub. Date: Apr. 9, 2009

(54) Injectable Particles

(75) Inventor: Robert E. Richard, Wrentham, MA (US)

Correspondence Address: MAYER & WILLIAMS PC, 251 NORTH AVENUE WEST, 2ND FLOOR, WESTFIELD, NJ, 07090, US

(73) Assignee: BOSTON SCIENTIFIC SCIMED, INC., Maple Grove, MN (US)

(21) Appl. No.: 12/244,996

(22) Filed: Oct. 3, 2008

Related U.S. Application Data
(60) Provisional application No. 60/997,829, filed on Oct. 5, 2007.

(51) Int. Cl. A61K 51/06; A61K 31/765

(52) U.S. Cl. 424/1.65; 424/78.31; 424/78.32

(57) ABSTRACT

According to an aspect of the invention, injectable polymeric particles are provided which contain branched poly(vinyl alcohol). Other aspects of the invention pertain to methods of making such particles. Still other aspects of the invention pertain to injectable compositions that comprise such particles and to methods of treatment that employ such injectable compositions.

### STATEMENT OF RELATED APPLICATIONS

This application claims the benefit of U.S. Provisional Patent Application Ser. No. 60/997,829, filed Oct. 5, 2007, entitled “Injectable Particles”, which is incorporated by reference herein in its entirety.

### FIELD OF THE INVENTION

The invention relates to particles for injection, more particularly, to polymeric injectable particles that comprise branched poly(vinyl alcohol).

### BACKGROUND OF THE INVENTION

Many clinical situations benefit from regulation of the vascular, lymphatic or duct systems by restricting the flow of body fluid or secretions. For example, the technique of embolization involves the introduction of particles into the circulation to occlude blood vessels, for example, so as to either arrest or prevent hemorrhaging or to cut off blood flow to a structure or organ. Permanent or temporary occlusion of blood vessels is desirable for managing various diseases and conditions.

In a typical embolization procedure, local anesthesia is first given over a common artery. The artery is then percutaneously punctured and a catheter is inserted and fluoroscopically guided into the area of interest. An angiogram is then performed by injecting contrast agent through the catheter. An embolic agent is then deposited through the catheter. The embolic agent is chosen, for example, based on the size of the vessel to be occluded, the desired duration of occlusion, and/or the type of disease or condition to be treated, among others factors. A follow-up angiogram is usually performed to determine the specificity and completeness of the arterial occlusion.

Various polymer-based microspheres are currently employed to embolize blood vessels. These microspheres are usually introduced to the location of the intended embolization through microcatheters. Many commercially available embolic microspheres are composed of polymers. Materials commonly used commercially for this purpose include polyvinyl alcohol (PVA), acetalized PVA (e.g., Contour SE™ embolic agent, Boston Scientific, Natick, Mass., USA) and crosslinked acrylic hydrogels (e.g., Embospheres®, Biosphere Medical, Rockland, Mass., USA). Similar microspheres have been used in chemoembolization to increase the residence time of the therapeutic after delivery. In one specific instance, a therapeutic agent (doxorubicin) has been directly added to polyvinyl alcohol hydrogel microspheres such that it can be released locally after delivery (e.g., DC Bead™ drug delivery chemoembolization system, Biocompatibles International plc, Farnham, Surrey, UK). Other examples of commercially available microspheres include glass microspheres with entrapped radioisotopes (e.g., ⁹⁰Y), in particular, TheraSpheres™, MDS Nordion, Ottowa, Canada and polymer microspheres that contain monomers that are capable of chelating radioisotopes (⁹⁰Y), in particular, SIR-Spheres®, SIRTex Medical, New South Wales, Australia.

It is also known to use polymer-based microspheres as augmentative materials for aesthetic improvement, including improvement of skin contour. Furthermore, polymer-based microspheres have also been used as augmentative materials in the treatment of various diseases, disorders and conditions, including urinary incontinence, vesicourethral reflux, fecal incontinence, intrinsic sphincter deficiency (ISD) and gastro-esophageal reflux disease. For instance, a common method for treating patients with urinary incontinence is via periurethral or transperineal injection of a bulking agent that contains polymer-based microspheres. In this regard, methods of injecting bulking agents for treatment of urinary incontinence commonly require the placement of a needle at a suitable treatment region, for example, periurethrally or transperineally. The bulking agent is injected into a plurality of locations, assisted by visual aids, causing the urethral lining to coapt.

### SUMMARY OF THE INVENTION

According to an aspect of the invention, injectable polymeric particles are provided that comprise branched poly(vinyl alcohol) particles.

Other aspects of the invention pertain to methods of making such particles.

Still other aspects of the invention pertain to injectable compositions that comprise such particles and to methods of treatment that employ such injectable compositions.

These and various additional aspects, embodiments and advantages of the present invention will become immediately apparent to those of ordinary skill in the art upon review of the Detailed Description and any claims to follow.

### DETAILED DESCRIPTION

In accordance with one aspect of the invention, injectable particles are provided which comprise branched poly(vinyl alcohol) (PVA). The injectable particles may be non-crosslinked or crosslinked via covalent and/or non-covalent means.

The injectable particles may be used to treat a variety of diseases and conditions in a variety of subjects. Subjects include vertebrate subjects, particularly humans and various warm-blooded animals including pets and livestock. As used herein, “treatment” refers to the prevention of a disease or condition, the reduction or elimination of symptoms associated with a disease or condition, or the substantial or complete elimination of a disease or condition.

The injectable particles of the invention may vary in shape. In certain embodiments, they are substantially spherical, for example, having the form of a perfect (to the eye) sphere or the form of a near-perfect sphere such as a prolate spheroid (a slightly elongated sphere) or an oblate spheroid (a slightly flattened sphere), among other regular or irregular near-spherical geometries.

In embodiments where the particles are substantially spherical, at least half of the particles (50% or more, for example, from 50% to 75% to 90% to 95% or more of a particle sample) may have a sphericity of 0.8 or more (e.g., from 0.80 to 0.85 to 0.9 to 0.95 to 0.97 or more). The sphericity of a collection of particles can be determined, for example, using a Beckman Coulter RapidVUE Image Analyzer version 2.06 (Beckman Coulter, Miami, Fla.). Briefly, the RapidVUE takes an image of continuous-tone (gray-scale) form and converts it to a digital form through the process of sampling and quantization. The system software identifies and measures the particles in an image. The sphericity of a particle, which is computed as Da/Dp (where Da=√(4A/π); Dp=P/π; A=pixel area; P=pixel perimeter), is a value from zero to one, with one representing a perfect circle.

The injectable particles of the invention can vary significantly in size, with typical longest linear cross-sectional dimensions (e.g., for a sphere, the diameter) ranging, for example, from 25 to 50 to 100 to 150 to 250 to 500 to 750 to 1000 to 1500 to 2000 to 2500 to 5000 microns (μm).

For a collection of particles, the arithmetic mean maximum for the group typically ranges, for example, from 40 to 100 to 150 to 250 to 500 to 750 to 1000 to 1500 to 2000 to 2500 to 5000 microns (μm). The arithmetic mean maximum dimension of a group of particles can be determined using a Beckman Coulter RapidVUE Image Analyzer version 2.06 (Beckman Coulter, Miami, Fla.), described above. The arithmetic mean maximum dimension of a group of particles (e.g., in a composition) can be determined by dividing the sum of the maximum dimensions (e.g., diameters for spherical particles) of all of the particles in the group by the number of particles in the group.

As used herein a “porous particle” is one that contains pores, which may be observed, for example, by viewing the injectable particles using a suitable microscopy technique such as scanning electron microscopy. Porous particles may be porous throughout or may comprise a non-porous core with a porous outer layer. Pore size may vary widely, ranging from 1 micron or less to 2 microns to 5 microns to 10 microns to 25 microns to 50 microns to 100 microns or more in width. Pores can be present in a wide range of shapes.

As used herein a “polymeric particle” is one that contains polymers, for example, from 50 wt % or less to 75 wt % to 90 wt % to 95 wt % to 97.5 wt % to 99 wt % or more polymers.

As used herein, “polymers” are molecules that contain multiple copies of one or more types of constitutional units, commonly referred to as monomers. The number of monomers/constitutional units within a given polymer may vary widely, ranging, for example, from 5 to 10 to 25 to 50 to 100 to 1000 to 10,000 or more constitutional units. As used herein, the term “monomers” may refer to the free monomers and those that are incorporated into polymers, with the distinction being clear from the context in which the term is used.

As used herein PVA is a polymer that comprises multiple vinyl alcohol monomers (—C—C(OH)—).

Branched PVA in accordance with the present invention may include monomers other than vinyl alcohol. As discussed further below examples of such monomers may include one or more of the following: vinyl ester monomers (e.g., vinyl acetate monomers), multifunctional monomers or macromers that correspond to branch points within the PVA, and monomers arising from chemical crosslinking, for example, vinyl formal monomers of the following structure,

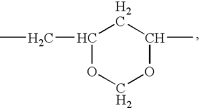

arising from an acetalization process, among others.

Branched PVA for use in the present invention can have a variety of architectures, including star-shaped architectures (e.g., architectures in which three or more chains emanate from a single branch point), comb architectures (e.g., architectures having a main chain and a plurality of side chains), and dendritic architectures (e.g., arborescent and hyperbranched polymers), among others.

In various embodiments, the branched PVA will have a lower radius of gyration compared to conventional (linear) poly(vinyl alcohol) at equivalent molecular weight. Radius of gyration may be measured by multi-angle light scattering.

In certain embodiments, the regions of polymeric material of the present invention may optionally contain supplemental polymers other than branched PVA.

In many embodiments of the invention, the injectable particles are hydrogel particles. As used herein, a “hydrogel” particle is a crosslinked polymer particle that swells when placed in water or biological fluids, but remains insoluble due to the presence of crosslinks, which may be, for example, physical, chemical, or both. For instance, a hydrogel particle in accordance with the invention may undergo swelling in water such that its longest linear cross-sectional dimension (e.g., for a sphere, the diameter) increases by 5% or less to 10% to 15% to 20% to 25% or more. In some embodiments, the insolubility of the hydrogel is not permanent, and the particles biodisintegrate in vivo.

In some embodiments, the injectable particle compositions in accordance with the invention further comprise one or more therapeutic agents. The therapeutic agents may be on, in and/or external to the particles, depending on the embodiment. “Therapeutic agents,” “biologically active agents,” “drugs,” “pharmaceutically active agents,” “pharmaceutically active materials,” and other related terms may be used interchangeably herein and include genetic therapeutic agents, non-genetic therapeutic agents and cells. Numerous therapeutic agents can be employed in conjunction with the present invention, including those used for the treatment of a wide variety of diseases and conditions (i.e., the prevention of a disease or condition, the reduction or elimination of symptoms associated with a disease or condition, or the substantial or complete elimination of a disease or condition). Numerous examples of therapeutic agents are described here.

Examples of therapeutic agents which may be used in the compositions of the invention for embolic applications include toxins (e.g., ricin toxin, radioisotopes, or any agents able to kill undesirable cells, such as those making up cancers and other tumors such as uterine fibroids) and agents that arrest growth of undesirable cells.

Specific examples of therapeutic agents may be selected from suitable members of the following: radioisotopes including ⁹⁰Y, ³²P, ¹⁸F, ¹⁴⁰La, ¹⁵³Sm, ¹⁶⁵Dy, ¹⁶⁶Ho, ¹⁶⁹Er, ¹⁶⁹Yb, ¹⁷⁷Lu, ¹⁸⁶Re, ¹⁸⁸Re, ¹⁰³Pd, ¹⁹⁸Au, ¹⁹²Ir, ⁹⁰Sr, ¹¹¹In or ⁶⁷Ga, antineoplastic/antiproliferative/anti-miotic agents including antimetabolites such as folic acid analogs/antagonists (e.g., methotrexate, etc.), purine analogs (e.g., 6-mercaptopurine, thioguanine, cladribine, which is a chlorinated purine nucleoside analog, etc.) and pyrimidine analogs (e.g., cytarabine, fluorouracil, etc.), alkaloids including taxanes (e.g., paclitaxel, docetaxel, etc.), alkylating agents such as alkyl sulfonates, nitrogen mustards (e.g., cyclophosphamide, ifosfamide, etc.), nitrosoureas, ethylenimines and methylmelamines, other aklyating agents (e.g., dacarbazine, etc.), antibiotics and analogs (e.g., daunorubicin, doxorubicin, idarubicin, mitomycin, bleomycins, plicamycin, etc.), platinum complexes (e.g., cisplatin, carboplatin, etc.), antineoplastic enzymes (e.g., asparaginase, etc.), agents affecting microtubule dynamics (e.g., vinblastine, vincristine, colchicine, Epo D, epothilone), caspase activators, proteasome inhibitors, angiogenesis inhibitors (e.g., statins such as endostatin, cerivastatin and angiostatin, squalamine, etc.), rapamycin (sirolimus) and its analogs (e.g., everolimus, tacrolimus, zotarolimus, etc.), etoposides, and many others (e.g., hydroxyurea, flavopiridol, procarbizine, mitoxantrone, campothecin, etc.), various pharmaceutically acceptable salts and derivatives (e.g., esters, etc.) of the foregoing, and combinations of the foregoing, among other agents.

Further therapeutic agents include chemical ablation agents (materials whose inclusion in the formulations of the present invention in effective amounts results in necrosis or shrinkage of nearby tissue upon injection) including osmotic-stress-generating agents (e.g., salts, etc.). Specific examples of chemical ablation agents from which suitable agents can be selected include the following: basic agents (e.g., sodium hydroxide, potassium hydroxide, etc.), acidic agents (e.g., acetic acid, formic acid, etc.), enzymes (e.g., collagenase, hyaluronidase, pronase, papain, etc.), free-radical generating agents (e.g., hydrogen peroxide, potassium peroxide, etc.), other oxidizing agents (e.g., sodium hypochlorite, etc.), tissue fixing agents (e.g., formaldehyde, acetaldehyde, glutaraldehyde, etc.), coagulants (e.g., gengpin, etc.), non-steroidal anti-inflammatory drugs, contraceptives (e.g., desogestrel, ethinyl estradiol, ethynodiol, ethynodiol diacetate, gestodene, lynestrenol, levonorgestrel, mestranol, medroxyprogesterone, norethindrone, norethynodrel, norgestimate, norgestrel, etc.), GnRH agonists (e.g, buserelin, cetorelix, decapeptyl, deslorelin, dioxalan derivatives, eulexin, ganirelix, gonadorelin hydrochloride, goserelin, goserelin acetate, histrelin, histrelin acetate, leuprolide, leuprolide acetate, leuprorelin, lutrelin, nafarelin, meterelin, triptorelin, etc.), antiprogestogens (e.g., mifepristone, etc.), selective progesterone receptor modulators (SPRMs) (e.g., asoprisnil, etc.), various pharmaceutically acceptable salts and derivatives of the foregoing, and combinations of the foregoing, among other agents.

For tissue bulking applications (e.g., urethral bulking, cosmetic bulking, etc.), specific beneficial therapeutic agents include those that promote collagen production, including proinflammatory agents and sclerosing agents such as those listed Pub. No. US 2006/0251697.

Proinflammatory agents can be selected, for example, from suitable endotoxins, cytokines, chemokines, prostaglandins, lipid mediators, and other mitogens. Specific examples of proinflammatory agents from which suitable agents can be selected include the following: growth factors such as platelet derived growth factor (PDGF), fibroblast growth factor (FGF), transforming growth factor (such as TGF-alpha and TGF-beta), epidermal growth factor (EGF), insulinlike growth factor (IGF), interleukins such as IL-1-(alpha or beta), IL-8, IL-4, IL6, IL-10 and IL-13, tumor necrosis factor (TNF) such as TNF-alpha, interferons such as INF-gamma, macrophage inflammatory protein-2 (MIP-2), leukotrienes such as leukotriene B4 (LTB4), granulocyte macrophage-colony stimulating factor (GM-CSF), cyclooxygenase-1, cyclooxygenase-2, macrophage chemotactic protein (MCP), inducible nitric oxide synthetase, macrophage inflammatory protein, tissue factor, phosphotyrosine phosphates, N-formyl peptides such as formyl-Met-Leu-Phe (fMLP), second mitochondria-derived activator of caspase (sMAC), activated complement fragments (C5a, C3a), phorbol ester (TPA), superoxide, hydrogen peroxide, zymosan, bacterial lipopolysaccharide, imiquimod, various pharmaceutically acceptable salts and derivates of the foregoing, and combinations of the foregoing, among other agents.

Suitable sclerosing agents for the practice of the invention can be selected, for example, from the following: inorganic materials such as aluminum hydroxide, sodium hydroxide, silver nitrate and sodium chloride, as well as organic compounds, including alcohols such as ethanol, acetic acid, trifluoroacetic acid, formaldehyde, dextrose, polyethylene glycol ethers (e.g., polidocanol, also known as laureth 9, polyethylene glycol (9) monododecyl ether, and hydroxypolyethoxydodecane), tetracycline, oxytetracycline, doxycycline, bleomycin, triamcinolone, minocycline, vincristine, iophendylate, tribenoside, sodium tetradecyl sulfate, sodium morrhuate, diatrizoate meglumine, prolamine diatrizoate, alkyl cyanoacrylates such as N-butyl-2-cyanoactyalte and methyl 2-cyanoacrylate, ethanolamine, ethanolamine oleate, bacterial preparations (e.g., corynebacterium and streptococcal preparations such as picibanil) and mixtures of the same, among others.

The amount of therapeutic agent within the compositions of the present invention will vary widely depending on a number of factors, including the disease or condition being treated, the potency of the therapeutic agent, and the volume of particulate composition that is ultimately injected into the subject, among other factors, with the therapeutically effective amount being readily determined by those of ordinary skill in the art. Where a therapeutic agent is provided within the compositions of the present invention, typical loadings range, for example, from 0.1 wt % or less, to 0.2 wt % to 0.5 wt % to 1 wt % to 2 wt % to 5 wt % to 10 wt % to 20 wt % or more of the dry weight of the composition.

In some embodiments, particles in accordance with the present invention may be provided with one or more binding groups that specifically or non-specifically interact with a therapeutic agent, for example, in order to retard or substantially eliminate the release of the therapeutic agent. For instance, such binding groups may be provided within the branched PVA or within a supplemental polymer other than PVA that may be present in the particles. Such binding groups may interact with the therapeutic agent via any of a variety of mechanisms, for example, based on non-covalent interactions such as van der Waals forces, hydrophobic interactions and/or electrostatic interactions (e.g., charge-charge interactions, charge-dipole interactions, and dipole-dipole interactions, including hydrogen bonding). Examples of specific non-covalent interactions include π-π stacking, binding based on the formation of multiple hydrogen bonds (e.g., polynucleotide hybridization, etc.), binding based on the formation of complexes and/or coordinative bonds (e.g., metal ion chelation, etc.), binding based on antibody-antigen interactions, also sometimes referred to as antibody-hapten interactions, protein-small molecule interactions (e.g., avidin/streptavidin-biotin binding), protein-protein interactions, and so forth.

As a specific example, where particulate compositions comprising radioisotopes are formed, it is desirable in some embodiments to provide the particles with one or more binding groups that interact with the radioisotopes via an electrostatic-based interaction such as ion exchange, complexation, coordination, chelation, etc. For instance, ligands such as acetylacetonates may be provided within the branched PVA (or within a supplemental polymer other than the branched PVA that may be present in the particles), which ligands are capable of forming coordination compounds (e.g., chelates) with charged radioactive ions. See, e.g., J. F. W. Nijsen et al., “Influence of neutron irradiation on holmium acetylacetonate loaded poly(L-lactic acid) microspheres,” Biomaterials, 23(8), April 2002, 1831-1839. An approach of this type is beneficial in that the polymers forming the particles need not be exposed to the high energy radiation that is associated with the conversion of non-radioactive species (e.g., ⁸⁹Y) to radioactive species (e.g., ⁹⁰Y). Instead, the particles can be loaded with the radioactive species after it is exposed to the high energy radiation. The exposure of most polymers to the levels of radiation needed to convert non-radioactive isotopes to radioactive would be expected to result in significant changes to the polymer (e.g., extensive chain scission and or crosslinking) resulting in modifications to the mechanical properties of the polymers, among other changes.

Branched PVA for use in the particles of the invention may be formed by any suitable method known in the art.

By way of background, the monomer of PVA (vinyl alcohol), does not exist in a stable free form, due to keto-enol rearrangement with its tautomer (acetaldehyde). Typically, PVA is produced by the polymerization of a vinyl ester such as vinyl acetate to form poly(vinyl acetate) (PVAc), followed by hydrolysis of PVAc to PVA. The hydrolysis reaction, however, does not typically go to completion, resulting in polymers with some degree of hydrolysis that depends on the extent of the hydrolysis reaction. Thus, PVA is generally a copolymer of vinyl alcohol and vinyl acetate. Commercial PVA grades are available with high degrees of hydrolysis (above 98.5%). The degree of hydrolysis (or, conversely, the acetate group content) of the polymer has an effect on its crystallizability and solubility, among other properties. For example, PVA grades having high degrees of hydrolysis are known to have reduced solubility in water relative to those having low degrees of hydrolysis. Moreover, PVA grades containing high degrees of hydrolysis are more difficult to crystallize relative to those having low degrees of hydrolysis. For further information on PVA (as well as PVA hydrogels), see, e.g., C. M. Hassan et al., “Structure and Applications of Poly(vinyl alcohol) Hydrogels Produced by Conventional Crosslinking or by Freezing/Thawing Methods,” Adv. Polym. Sci., 153, 37-65 (2000) and N. A. Peppas et al., “Hydrogels in Biology and Medicine: From Fundamentals to Bionanotechnology”, Adv. Mater., 18, 1345-1360 (2006).

Methods of forming branched PVA polymers include methods that employ multifunctional monomers to create branch points within the PVA. For example, R. Baudry et al., Macromolecules, 2006, 39, 5230-5237 describe a technique in which branched PVA polymers are formed via free radical copolymerization of vinyl acetate (VAc) and a trifunctional monomer (triallyl-triazine-trione) (TTT) in 2-isopropoxy ethanol (IPE) solvent using azobisisobutyronitrile) (AlBN) as an initiator in the presence of a suitable thiol free-radical chain-transfer agent (RSH), specifically 2-mercaptoethanol, 3-mercaptopropane-1,2-diol or di(2-mercaptoethyl)ether), to inhibit crosslinking. The reaction scheme is represented as follows:

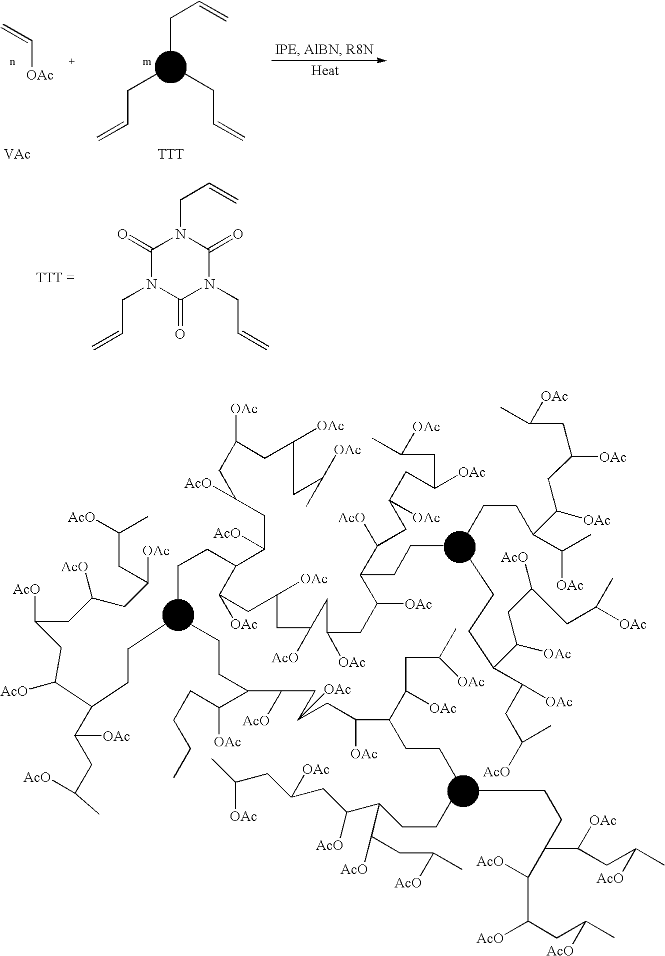

The thus-formed branched PVAc polymers were then subjected to alcoholysis using methanol. A high level of conversion of acetate to hydroxyl groups was reported.

As another example, J. Bernard et al., Polymer, 47(4) 2006, 1073-1080, report the preparation of poly(vinyl alcohol) comb-type polymers. Poly(vinyl acetate) (PVAc) branches were prepared via interchange of xanthate (MADIX)/reversible addition-fragmentation chain-transfer (RAFT) polymerization using xanthate functionalized PVA backbones as macromers. Controlled radical polymerization reactions such as RAFT offer good control over polymer architecture, polydispersity and molecular weight. The ester linkages between the PVAc branches and the PVA backbone were sufficiently stable to allow hydrolysis of the PVAc branches, yielding poly(vinyl alcohol) combs.

As indicated above, hydrogels are crosslinked hydrophilic polymers (e.g., polymer networks) which swell when placed in water or biological fluids, but remain insoluble due to the presence of crosslinks, which may be, for example, physical, chemical, or both.

Branched PVA particles may be crosslinked, for example, through the use of mono-functional and multifunctional chemical crosslinking agents. For instance, branched PVA may be crosslinked through the use of monoaldehydes such as acetaldehyde or formaldehyde, or dialdehydes such as glutaraldehyde, among others. In the presence of an acid such as sulfuric acid or acetic acid, these crosslinking agents form acetal bridges between the pendant hydroxyl groups found on the polymer chains. For example, acetal formation may proceed to link two alcohol moieties together according to the following scheme:

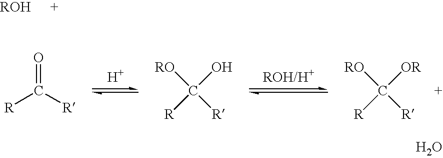

where R and R′ are organic groups. For species with multiple hydroxyl groups, including polyols such as PVA, two hydroxyl groups within the same molecule may react according to the following scheme:

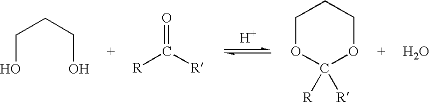

As noted in Pub. No. US 2003/0185895 to Lanphere et al., the reaction of linear PVA with an aldehyde (formaldehyde) in the presence of an acid is primarily a 1,3 acetalization reaction:

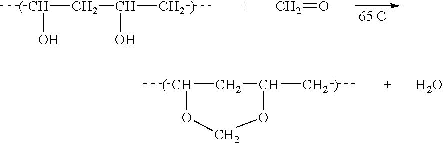

Since the reaction proceeds in a random fashion, there are leftover —OH groups that do not react with adjacent groups.

Other methods which may be used to crosslink branched PVA particles include electron-beam and gamma-ray irradiation. These methods, as well as physical crosslinking techniques such as freeze/thaw processing, may in some instances be advantageous over techniques that employ chemical cross-linking agents, because they do not leave behind unreacted chemical species.

As a specific example, microspheres of PVA suitable for injection (e.g., for embolic, bulking or other purposes) can be prepared by dispersing an aqueous PVA solution in an immiscible solvent and then crosslinking it with a suitable material such as an aldehyde.

As another specific example, PVA microspheres may be formed using a modified version of the process described in Pub. No. US 2003/0185895 to Lanphere et al. For instance, a solution containing a branched PVA and a gelling precursor such as sodium alginate may be delivered to a viscosity controller, which heats the solution to reduce its viscosity prior to delivery to a drop generator. The drop generator forms and directs drops into a gelling solution containing a gelling agent which interacts with the gelling precursor. For example, in the case where an alginate gelling precursor is employed, an agent containing a divalent metal cation such as calcium chloride may be used as a gelling agent, which stabilizes the drops by gel formation based on ionic crosslinking. The gel-stabilized drops may then be transferred to a reactor vessel where the branched PVA in the gel-stabilized drops is crosslinked. For example, the reactor vessel may include an agent that chemically reacts with the branched PVA to cause interchain and/or intrachain crosslinking. For instance, the vessel may include an aldehyde and an acid, leading to acetalization of the branched PVA. The precursor particles are then transferred to a gel dissolution chamber, where the gel is dissolved. For example, ionically crosslinked alginate may be removed by ion exchange with a solution of sodium hexa-metaphosphate. Alginate may also be removed by radiation degradation. Porosity is generated due to the presence (and ultimate removal) of the alginate. The particles may then be filtered to remove any residual debris and to sort the particles into desired size ranges. Other particle formation/crosslinking techniques may be employed as well.

Due to the viscosity of the linear PVA (and alginate), there is currently a specific limit to the concentration of PVA that can be used in the above process described in Lanphere et al. Branched PVA, on the other hand, has a lower viscosity than the common linear PVA, allowing for more concentrated PVA solutions to be used in the formation of the particles of the invention. Consequently, by using branched PVA, microspheres may be formed having increased density relative to those formed with linear PVA. In addition, when the branched PVA is reacted with the aldehyde in the acetalization step, the highly branched nature of the PVA may expand the type (e.g., intrachain vs. interchain) and degree of crosslinking that can be achieved. Moreover, highly branched PVA may respond differently to radiation, resulting in higher radiation-derived crosslink density. Thus, by using branched PVA, rather than linear PVA, microspheres may be formed, for example, which have unique characteristics selected from one or more of the following: morphology, density, compressibility, pore size and distribution, type and density of crosslinking, therapeutic agent loading and delivery characteristics (where a therapeutic agent is present), and radiation resistance, among others.

If desired, one or more optional agents such as therapeutic agents can be incorporated at various stages of the production process. For example, injectable microparticles in accordance with the invention can be placed in a solution that includes a therapeutic agent. In some embodiments, the particles are dried by a suitable method, for example, by lyophilization (freeze drying), prior to placing them in the therapeutic-agent-containing solution. In the rehydration process, the therapeutic agent is drawn into the particles. The particle composition may be re-dried at this stage, if desired.

As another example, a therapeutic agent may be added during formation of the PVA-containing polymeric region. For instance, a therapeutic agent may be mixed with the branched PVA and alginate prior to gel formation, among numerous other possibilities.

As another example, a therapeutic agent may be added by a medical practitioner to a particulate composition in accordance with the invention at the time of administration to a subject.

The particle compositions of the invention may be stored and transported in a sterile dry form. The dry composition may also optionally contain additional agents, for example, one or more of the following among others: (a) tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, (b) suspension agents including various surfactants, wetting agents, and polymers (e.g., albumen, PEO, polyvinyl alcohol, block copolymers, etc.), among others, (c) imaging contrast agents (e.g., Omnipaque™, Visipaque™, etc.), (d) pH adjusting agents including various buffer solutes, and (e) therapeutic agents. The dry composition may shipped, for example, in a syringe, catheter, vial, ampoule, or other container, and it may be mixed with a suitable liquid carrier (e.g. sterile water for injection, physiological saline, phosphate buffer, a solution containing an imaging contrast agent, etc.) prior to administration. In this way the concentration of the composition to be injected may be varied at will, depending on the specific application at hand, as desired by the health care practitioner in charge of the procedure. One or more containers of liquid carrier may also be supplied and shipped, along with the dry particles, in the form of a kit.

The injectable particles may also be stored in a sterile suspension that contains water in addition to the particles themselves, as well as other optional agents such as one or more of the tonicity adjusting agents, suspension agents, contrast media, pH adjusting agents, and therapeutic agents listed above, among others. The suspension may be stored, for example, in a syringe, catheter, vial, ampoule, or other container. The suspension may also be mixed with a suitable liquid carrier (e.g. sterile water for injection, physiological saline, phosphate buffer, a solution containing contrast agent, etc.) prior to administration, allowing the concentration of administered particles (as well as other optional agents) in the suspension to be reduced prior to injection, if so desired by the health care practitioner in charge of the procedure. One or more containers of liquid carrier may also be supplied to form a kit.

The amount of injectable particles within a suspension to be injected may be determined by those of ordinary skill in the art. The amount of particles may be limited by the fact that when the amount of particles in the composition is too low, too much liquid may be injected, possibly allowing particles to stray far from the site of injection, which may result in undesired embolization or bulking of vital organs and tissues. When the amount of particles is too great, the delivery device (e.g., catheter, syringe, etc.) may become clogged.

An effective amount of the particle compositions of the invention is, for example, (a) an amount sufficient to produce an occlusion or emboli at a desired site in the body, (b) an amount sufficient to achieve the degree of bulking desired (e.g., an amount sufficient to improve urinary incontinence, vesicourethral reflux, fecal incontinence, ISD or gastro-esophageal reflux, or an amount sufficient for aesthetic improvement), or (c) an amount sufficient to locally treat a disease or condition. Effective doses may also be extrapolated from dose-response curves derived from animal model test systems, among other techniques.

In certain embodiments, the density of the aqueous phase that suspends the particles is close to that of the particles themselves, thereby promoting an even suspension. The density of the aqueous phase may be increased, for example, by increasing the amount of solutes that are dissolved in the aqueous phase, and vice versa.

As noted above, permanent or temporary occlusion of blood vessels is useful for managing various diseases and conditions. For example, fibroids, also known as leiomyoma, leiomyomata or fibromyoma, are the most common benign tumors of the uterus. These non-cancerous growths are present in significant fraction of women over the age of 35. In most cases, multiple fibroids are present, often up to 50 or more. Fibroids can grow, for example, within the uterine wall (“intramural” type), on the outside of he uterus (“subserosal” type), inside the uterine cavity (“submucosal” type), between the layers of broad ligament supporting the uterus (“interligamentous” type), attached to another organ (“parasitic” type), or on a mushroom-like stalk (“pedunculated” type). Fibroids may range widely in size, for example, from a few millimeters to 40 centimeters. In some women, fibroids can become enlarged and cause excessive bleeding and pain. While fibroids have been treated in the past by surgical removal of the fibroids (myomectomy) or by removal of the uterus (hysterectomy), recent advances in uterine embolization now offer a nonsurgical treatment. Thus, injectable compositions in accordance with the present invention can be used to treat uterine fibroids.

Methods for treatment of fibroids by embolization are well known to those skilled in the art (see, e.g., Pub. No. US 2003/0206864 to Mangin and the references cited therein). Uterine embolization is aimed at starving fibroids of nutrients. Numerous branches of the uterine artery may supply uterine fibroids. In the treatment of fibroids, embolization of the entire uterine arterial distribution network is often preferred. This is because it is difficult to selectively catheterize individual vessels supplying only fibroids, the major reason being that there are too many branches for catheterization and embolization to be performed in an efficient and timely manner. Also, it is difficult to tell whether any one vessel supplies fibroids rather than normal myometrium. In many women, the fibroids of the uterus are diffuse, and embolization of the entire uterine arterial distribution affords a global treatment for every fibroid in the uterus.

In a typical procedure, a catheter is inserted near the uterine artery by the physician (e.g., with the assistance of a guide wire). Once the catheter is in place, the guide wire is removed and contrast agent is injected into the uterine artery. The patient is then subjected to fluoroscopy or X-rays. In order to create an occlusion, an embolic agent is introduced into the uterine artery via catheter. The embolic agent is carried by the blood flow in the uterine artery to the vessels that supply the fibroid. The particles flow into these vessels and clog them, thus disrupting the blood supply to the fibroid. In order for the physician to view and follow the occlusion process, contrast agent may be injected subsequent to infusion of the embolic agent. Treatment may be enhanced in the present invention by including a therapeutic agent (e.g., antineoplastic/antiproliferative/anti-miotic agent, toxin, ablation agent, etc.) in the particulate composition.

Controlled, selective obliteration of the blood supply to tumors is also used in treating solid tumors such as renal carcinoma, bone tumor and liver cancer, among various others. The idea behind this treatment is that preferential blood flow toward a tumor will carry the embolization agent to the tumor thereby blocking the flow of blood which supplies nutrients to the tumor, causing it to shrink. Embolization may be conducted as an enhancement to chemotherapy or radiation therapy. Treatment may be enhanced in the present invention by including a therapeutic agent (e.g., antineoplastic/antiproliferative/anti-miotic agent, toxin, ablation agent, etc.) in the particulate composition.

Particle compositions in accordance with the invention may also be used to treat various other diseases, conditions and disorders, including treatment of the following: arteriovenous fistulas and malformations including, for example, aneurysms such as neurovascular and aortic aneurysms, pulmonary artery pseudoaneurysms, intracerebral arteriovenous fistula, cavernous sinus dural arteriovenous fistula and arterioportal fistula, chronic venous insufficiency, varicocele, pelvic congestion syndrome, gastrointestinal bleeding, renal bleeding, urinary bleeding, varicose bleeding, uterine hemorrhage, and severe bleeding from the nose (epistaxis), as well as preoperative embolization (to reduce the amount of bleeding during a surgical procedure) and occlusion of saphenous vein side branches in a saphenous bypass graft procedure, among other uses. As elsewhere herein, treatment may be enhanced in the present invention by including a therapeutic agent in the particulate composition.

Particle compositions in accordance with the invention may also be used in tissue bulking applications, for example, as augmentative materials in the treatment of urinary incontinence, vesicourethral reflux, fecal incontinence, intrinsic sphincter deficiency (ISD) or gastro-esophageal reflux disease, or as augmentative materials for aesthetic improvement. For instance, a common method for treating patients with urinary incontinence is via periurethral or transperineal injection of a bulking material. In this regard, methods of injecting bulking agents commonly require the placement of a needle at a treatment region, for example, periurethrally or transperineally. The bulking agent is injected into a plurality of locations, assisted by visual aids, causing the urethral lining to coapt. In some cases, additional applications of bulking agent may be required. Treatment may be enhanced by including a therapeutic agent (e.g., proinflammatory agents, sclerosing agents, etc.) in the particulate composition.

The present invention encompasses various ways of administering the particulate compositions of the invention to effect embolization, bulking or other procedure benefiting from therapeutic agent release. One skilled in the art can determine the most desirable way of administering the particles depending on the type of treatment and the condition of the patient, among other factors. Methods of administration include, for example, percutaneous techniques as well as other effective routes of administration. For example, the particulate compositions of the invention may be delivered, for example, through a syringe or through a catheter, for instance, a Tracker® microcatheter (Boston Scientific, Natick, Mass., USA), which can be advanced over a guidewire, a steerable microcatheter, or a flow-directed microcatheter (MAGIC, Balt, Montomorency, France).

Various aspects of the invention of the invention relating to the above are enumerated in the following paragraphs:

Aspect 1. Injectable polymeric particles comprising branched poly(vinyl alcohol).

Aspect 2. The injectable polymeric particles of Aspect 1, wherein the branched poly(vinyl alcohol) comprises a multifunctional monomer.

Aspect 3. The injectable polymeric particles of Aspect 2, wherein the multifunctional monomer is triallyl-triazine-trione.

Aspect 4. The injectable polymeric particles of Aspect 1, wherein the branched poly(vinyl alcohol) has a lower radius of gyration compared to conventional poly(vinyl alcohol) at equivalent molecular weight.

Aspect 5. The injectable polymeric particles of Aspect 1, wherein the branched poly(vinyl alcohol) has a comb architecture.

Aspect 6. The injectable polymeric particles of Aspect 1, wherein the branched poly(vinyl alcohol) comprise at least 50% vinyl alcohol monomers.

Aspect 7. The injectable polymeric particles of Aspect 1, wherein the injectable polymeric particles are crosslinked.

Aspect 8. The injectable polymeric particles of Aspect 7, wherein the branched poly(vinyl alcohol) is covalently crosslinked by intrachain crosslinking, interchain crosslinking, or both.

Aspect 9. The injectable polymeric particles of Aspect 7, wherein the injectable polymeric particles comprise crosslinks that comprise acetal linkages.

Aspect 10. The injectable polymeric particles of Aspect 1, wherein the arithmetic mean maximum dimension is between 40 μm and 5000 μm.

Aspect 11. The injectable polymeric particles of Aspect 1, wherein at least half of the particles have a sphericity of 0.8 or more.

Aspect 12. The injectable polymeric particles of Aspect 1, wherein the particles are porous particles.

Aspect 13. The injectable polymeric particles of Aspect 1, wherein the particles are biostable.

Aspect 14. The injectable polymeric particles of Aspect 1, wherein the particles are hydrogel particles.

Aspect 15. The injectable polymeric particles of Aspect 1, further comprising a therapeutic agent selected from toxins, antineoplastic agents, ablation agents, proinflammatory agents and sclerosing agents.

Aspect 16. The injectable polymeric particles of Aspect 1, further comprising a therapeutic agent that non-covalently binds to the particles by electrostatic interactions with binding groups in the particles.

Aspect 17. The injectable polymeric particles of Aspect 16, wherein the therapeutic agent is a charged radioisotope and the particles comprise ligands that form coordination complexes with the charged radioisotope.

Aspect 18. An injectable medical composition comprising the particles of Aspect 1.

Aspect 19. The injectable medical composition of Aspect 18, comprising a tonicity adjusting agent.

Aspect 20. The injectable medical composition of Aspect 19, wherein the tonicity adjusting agent is selected from sugars, polyhydric alcohols, inorganic salts and combinations thereof.

Aspect 21. The injectable medical composition of Aspect 18, wherein the injectable medical composition is disposed within a glass container or a preloaded medical device.

Aspect 22. A method of forming the injectable polymeric particles of Aspect 8, comprising forming polymeric particles that comprises branched poly(vinyl alcohol) and exposing the polymeric particles to a covalent crosslinking agent.

Aspect 23. The method of Aspect 22, wherein the covalent crosslinking agent is an aldehyde crosslinking agent.

Although various aspects and embodiments are specifically illustrated and described herein, it will be appreciated that modifications and variations of the present invention are covered by the above teachings and are within the purview of any appended claims without departing from the spirit and intended scope of the invention.

1. Injectable polymeric particles comprising branched poly(vinyl alcohol).

2. The injectable polymeric particles of claim 1, wherein said branched poly(vinyl alcohol) comprises a multifunctional monomer.

3. The injectable polymeric particles of claim 2, wherein said multifunctional monomer is triallyl-triazine-trione.

4. The injectable polymeric particles of claim 1, wherein said branched poly(vinyl alcohol) has a lower radius of gyration compared to conventional poly(vinyl alcohol) at equivalent molecular weight.

5. The injectable polymeric particles of claim 1, wherein said branched poly(vinyl alcohol) has a comb architecture.

6. The injectable polymeric particles of claim 1, wherein said branched poly(vinyl alcohol) comprise at least 50% vinyl alcohol monomers.

7. The injectable polymeric particles of claim 1, wherein said injectable polymeric particles are crosslinked.

8. The injectable polymeric particles of claim 7, wherein said branched poly(vinyl alcohol) is covalently crosslinked by intrachain crosslinking, interchain crosslinking, or both.

9. The injectable polymeric particles of claim 7, wherein said injectable polymeric particles comprise crosslinks that comprise acetal linkages.

10. The injectable polymeric particles of claim 1, wherein the arithmetic mean maximum dimension is between 40 μm and 5000 μm.

11. The injectable polymeric particles of claim 1, wherein at least half of said particles have a sphericity of 0.8 or more.

12. The injectable polymeric particles of claim 1, wherein said particles are porous particles.

13. The injectable polymeric particles of claim 1, wherein said particles are biostable.

14. The injectable polymeric particles of claim 1, wherein said particles are hydrogel particles.

15. The injectable polymeric particles of claim 1, further comprising a therapeutic agent selected from toxins, antineoplastic agents, ablation agents, proinflammatory agents and sclerosing agents.

16. The injectable polymeric particles of claim 1, further comprising a therapeutic agent that non-covalently binds to the particles by electrostatic interactions with binding groups in the particles.

17. The injectable polymeric particles of claim 16, wherein said therapeutic agent is a charged radioisotope and the particles comprise ligands that form coordination complexes with the charged radioisotope.

18. An injectable medical composition comprising said particles of claim 1.

19. The injectable medical composition of claim 18, comprising a tonicity adjusting agent.

20. The injectable medical composition of claim 19, wherein said tonicity adjusting agent is selected from sugars, polyhydric alcohols, inorganic salts and combinations thereof.

21. The injectable medical composition of claim 18, wherein said injectable medical composition is disposed within a glass container or a preloaded medical device.

22. A method of forming the injectable polymeric particles of claim 8, comprising forming polymeric particles that comprises branched poly(vinyl alcohol) and exposing the polymeric particles to a covalent crosslinking agent.

23. The method of claim 22, wherein said covalent crosslinking agent is an aldehyde crosslinking agent.
